(19) United States
(12) Patent Application Publication
(10) Pub. No.: US 2022/0225933 A1
(43) Pub. Date: Jul. 21, 2022

(54) Smart Band For Alerting During Emergencies

(71) Applicant: Annabelle Rehn, Kuala Lumpur (MY)

(72) Inventor: Annabelle Rehn, Kuala Lumpur (MY)

(21) Appl. No.: 17/608,987

(22) PCT Filed: May 5, 2020
(86) PCT No.: PCT/IB2020/054268

§ 371 Date: Nov. 4, 2021

Related U.S. Application Data
(60) Provisional application No. 62/843,483, filed on May 5, 2019.

(51) Int. Cl. A61B 5/00; A61B 5/024; G08B 21/04; G08B 25/00; G08B 25/01; G08B 7/06; H04W 4/90; H04W 4/029

(52) U.S. Cl. CPC A61B 5/681; A61B 5/742; A61B 5/746; A61B 5/747; A61B 5/7405; H04W 4/029; G08B 21/0453; G08B 25/001; G08B 25/016; G08B 7/06; H04W 4/90; A61B 5/02438

(57) ABSTRACT

A smart band, for alerting of a user in an emergency, includes a bracelet to be worn by the user. A screen is disposed along the bracelet to display alerts and messages. An alert system notifies that the user is in an emergency. The screen includes technology compatible with an application on a phone or a computer. A GPS tracker identifies and displays a location of the user. The bracelet includes a back surface having a barcode to identify the bracelet. A locking mechanism prevents the user from removing the bracelet. The locking mechanism is unlocked by a tool to allow the bracelet to be removed.

### FIELD OF THE INVENTION

The present invention relates to a smart band for identifying emergencies during swimming activities based on heart rate irregularities and sending alerts to the appropriate individuals.

### BACKGROUND OF THE INVENTION

Every year, there are approximately 4.5 million cases of unintentional drowning, resulting in 324,000 deaths. Among children ages 1-14, drowning is the second leading cause of unintentional injury-related deaths. Life vests and other safety measures have been put in place to prevent drowning. However, these measures have proven insufficient in preventing deaths.

The present invention resolves the current problems via a waterproof smart band worn by a child, comprising technology that measures heart rate irregularities. The smart band is compatible with an application and provides real-time alerts of a potential drowning situation.

### BRIEF SUMMARY OF THE EMBODIMENTS OF THE INVENTION

A smart band, for alerting of a user in an emergency, includes a bracelet to be worn by the user. A screen is disposed along the bracelet to display alerts and messages. An alert system notifies that the user is in an emergency. The screen includes technology compatible with an application on a phone or a computer. A GPS tracker identifies and displays a location of the user. The bracelet includes a back surface having a barcode to identify the bracelet. A locking mechanism prevents the user from removing the bracelet. The locking mechanism is unlocked by a tool to allow the bracelet to be removed.

In a variant, the smart band comprises a bracelet to be worn by the user; a screen disposed along the bracelet to display alerts and messages; and an alert system for notifying that the user is in an emergency. The screen is powered by a battery having a battery life. The smart band includes technology compatible with an application on a phone or a computer or an external computing device. The smart band has a sensor for measuring heart rate. The smart band is configured to monitor a heart rate via a processor that executes instructions stored on a non-transitory computer readable medium, based on measurements received by the heart rate sensor, and analyze the measurements and determine whether a user is in a state of emergency and then send an alert if the user is in a state of emergency.

In another variant, the smart band comprises a plurality of LED lights, disposed on the screen, that when lit, correspond to a percentage of the battery life of the battery. The screen displays a warning message when the percentage of the battery life drops to a particular amount. The screen is configured to indicate how much time the battery is operable the battery being connectable to a charger for replenishing the battery life of the battery.

In a further variant, the smart band comprises a first list of identifying personal contacts and a second list of identifying emergency contacts, both stored on the band and displayable on the screen. The first list and the second list connect to a personal application and a corporate application, respectively, on a separate device.

In still another variant, the smart band has an alarm siren and a single LED light synced to both the personal application and the corporate application. The alarm siren is louder and more visible than the alert system, and provides a sound to be heard by a bystander during the emergency.

In yet a further variant of the smart band, both the alarm siren and the single LED light are powered a plurality of batteries separate from the battery.

In a variant, the bracelet has a back surface having a barcode to identify the bracelet, and a locking mechanism that prevents the user from removing the bracelet. The locking mechanism configured to be unlocked by a tool to allow the bracelet to be removed.

In another variant, the smart band is configured to analyze the heart rate measurements and compare the measurements to a predetermined baseline value and a heart rate zone within which the user may be at risk; wherein the smart band is configured to send an alert to an external computing device for alerting another individual, if the heart rate measurement is sustained within the zone for at least a predetermined period of time.

In a further variant, the smart band is configured to determine if there is confirmation of receipt of an alert sent to the external computing device, and the smart band also determines if a recipient confirms or does not confirm receipt of the alert within a predetermined period of time of the alert; wherein if no confirmation takes place, then the smart band instructs a server to send a phone call and a text message to the another individual; wherein if confirmation does take place, then the smart band validates the alert system of the smart band.

In still another variant, the smart band has a GPS tracker for identifying and displaying a location of the user to the computing device of the another individual. The smart band is configured to send the location of the user based on data provided by the GPS tacker.

In yet a further variant, a smart band for alerting of a user in an emergency, comprises: a bracelet to be worn by the user; a screen disposed along the bracelet to display alerts and messages, powered by a battery having a battery life, and wherein the screen displays a warning message when the percentage of the battery life drops to a particular amount, and the screen is configured to indicate how much time the battery is operable the battery being connectable to a charger for replenishing the battery life of the battery. The smart band has a first list identifying personal contacts and a second list identifying emergency contacts, both stored on the screen or band. The first list and the second list connect to a personal application and a corporate application, respectively, on a separate device. The smart band has a sensor for measuring heart rate; a plurality of LED lights, disposed on the screen, that when lit, correspond to a percentage of the battery life of the battery; and an alert system for notifying that the user is in an emergency. The band includes technology compatible with an application on an external computing device and has an alarm siren and a single LED light synced to both the personal application and the corporate application. The alarm siren is louder and more visible than the alert system, and provides a sound to be heard by a bystander during the emergency. The smart band has a GPS tracker for identifying and displaying a location of the user to the computing device of the another individual. The smart band is configured to send the location of the user based on data provided by the GPS tacker. The smart band is configured to monitor a heart rate via a processor that executes instructions stored on a non-transitory computer readable medium, based on measurements received by the heart rate sensor, and analyze the measurements and determine whether a user is in a state of emergency and then send an alert if the user is in a state of emergency. The smart band is configured to analyze the heart rate measurements and compare the measurements to a predetermined baseline value and a heart rate zone within which the user may be at risk; wherein the smart band is configured to send an alert to an external computing device for alerting another individual, if the heart rate measurement is sustained within the zone for at least a predetermined period of time. The smart band is configured to determine if there is confirmation of receipt of an alert sent to the external computing device, and the smart band also determines if a recipient confirms or does not confirm receipt of the alert within a predetermined period of time of the alert. If no confirmation takes place, then the smart band instructs a server to send a phone call and a text message to the another individual. If confirmation does take place, then the smart band validates the alert system of the smart band. Both the alarm siren and the single LED light are powered a plurality of batteries separate from the battery. The bracelet has a back surface having a barcode to identify the bracelet, and a locking mechanism that prevents the user from removing the bracelet; wherein the locking mechanism is configured to be unlocked by a tool to allow the bracelet to be removed.

In a variant, a method for determining if a user is in an unsafe situation comprises the steps of: receiving measurements of a heart rate from the user; analyzing the measurements; comparing the measurements to a baseline value; and alerting the user and other recipients when the measurements include a value within a range higher or lower than the baseline value.

In another variant, the method comprises confirming receipt of an initial alert by the other recipients by selecting a prompt that appears on a screen of an external device.

In a further variant, the method comprises the steps of sending a phone call and a text message to the other recipients if receipt of the initial alert is not confirmed within a predetermined period of time of the initial alert.

In still another variant of the method, alerting the user and other recipients when the measurements include a value within a range higher or lower than the baseline value, further comprises: utilizing machine learning techniques to validate the measurements as being an accurate basis for determining the unsafe conditions.

In yet another variant of the method, the unsafe conditions comprise: drowning, abnormal heart rates, abnormal breathing rates, or a combination thereof.

### BRIEF DESCRIPTION OF THE DRAWINGS

FIG. 1 is a block diagram of a smart bracelet interfacing with multiple external systems.

FIG. 2 is a block diagram of a server with multiple components interfacing with multiple external systems.

FIG. 3 is a flowchart, wherein the steps of the flowchart are performed by the smart bracelet.

FIG. 4 is a block diagram of a computing device, which can be used for implementing the present invention.

### DETAILED DESCRIPTION OF THE EMBODIMENTS OF THE INVENTION

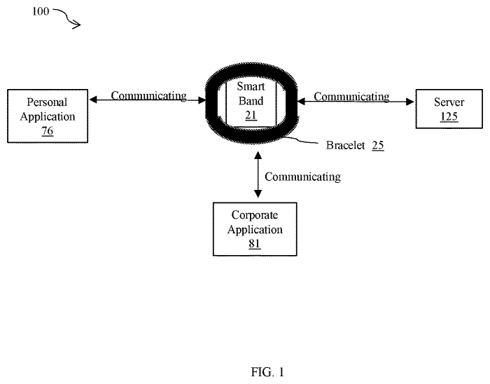

In an embodiment, depicted in FIG. 1, a network 100 includes and connects a personal application 76, a corporate application 81, a server 125, and a smart band 21. The personal application 76 includes a first respondent contact (e.g. a parent, a guardian, or a supervisor) and a second respondent contact, or an emergency contact (e.g. a relative or a close friend who lives close by and can contact the first respondent contact in an emergency). For the corporate application 81, a supervisor can fill in contact information for a user wearing the smart band 21, or contact a manager of a location (e.g. a waterpark) on a separate device. The network 100 is a digital telecommunications network for sharing resources between nodes (i.e., computing devices). Data transmission between nodes is supported over physical connections (twisted pair and fiber-optic cables) and/or wireless connections (Wi-Fi, microwave transmission, and free-space optical communication). The smart band 21 contains a computer program that connects to a GPS tracker module, lights for a light emitting device (LED), networking interfacing modules that connect to the server 125, the personal application 76, and the corporate application 81, database(s), and machine learning (ML) algorithms. The personal application 76 and the corporate application 81 both reside in a programmable computing device or external computing device, which is instructed to carry out sequences of arithmetic or logical operations via computer programming. The programmable computing device may include without limitation a Smartphone device, a tablet computer, a personal computer, a laptop computer, a terminal device, and a cellular phone.

The smart band 21 (as illustrated in FIG. 1) for alerting an individual to an emergency, includes a bracelet 25 to be worn by a user, by sending the alert to the individual's computing device. An alert system (not shown) of the smart band 21 notifies the individual that the user is in an emergency. A screen (not shown) is disposed along the bracelet 25 to display alerts and messages. The screen is powered by a battery (not shown) having a battery life, wherein the battery life refers to how much time the battery is operable. A plurality of LED lights is disposed on, or along, the screen. The LED lights, when lit, correspond to a percentage of the battery life of the battery. The battery connects to a charger (not shown). The charger replenishes the battery life of the battery. A GPS tracker module (not shown) in the smart band 21 identifies and displays a location of the user. The screen includes technology compatible with an application on a phone or a computer. The GPS tracker module communicates via cellular technologies, that are available in the location. Communication takes place between the smart band 21 on the user and a device of the individual. For instance, if the user is in water, the GPS tracker module communicates up to a maximum depth of 3 meters. The location is accessed in real time when the user is in a more open and crowded venue, such as a water park or a beach. The screen displays a warning message when the percentage of the battery life drops to a particular amount, for instance 25%. The percentage of the batter life remaining is displayed on the screen of the smart band 21. When the battery is fully charged, an indication is displayed on the screen of the smart band 21. The bracelet 25 includes a back surface (not shown) that has a barcode (not shown) to identify the bracelet 25. The bracelet 25 is available in various colors. The bracelet 25 includes a locking mechanism (not shown) that prevents the user (typically a child) from removing the bracelet 25. The locking mechanism is unlocked by a tool to allow the bracelet 25 to be removed by an adult teacher, parent, or caregiver.

In another embodiment, the smart band 21 includes a number of features to categorize different individuals. For example, the smart band 21 identifies personal contacts in a first list and emergency contacts in a second (corporate) list, stored on the screen. Each of these lists connects to a personal application 76 and a corporate application 81, respectively, on a device such as a phone or a computer. Multiple users may be created on one of the applications, but all of the users are visible on the screen.

In a further embodiment, the smart band 21 includes an alarm siren (not shown) and a single LED light (not shown) synced to both the personal application 76 and the corporate application 81. The alarm siren and the single LED light are paired with the smart band 21, as well as with the device of the individual (i.e. a phone). When the smart band 21 recognizes a potential drowning situation, an initial alert is sent to the device of the individual through a cellular connection of up to 3 meters of depth in any type of body of water (e.g. salt water, chlorinated water, fresh water). A signal is sent to the device of the individual with a countdown timer of a short predetermined period of time (for example, 5 seconds), only if the alarm siren and the single LED light are both paired with the device of the individual. This allows the individual to activate or deactivate the alarm siren in the event of a faulty reading. If the signal is not responded to within the predetermined period of time (for example, 5 seconds), the alarm siren and the single LED light will start automatically. In the event that the individual chooses to activate the alarm siren, but deactivates the alarm siren due to panicking, a message is displayed asking to confirm the decision to activate or deactivate. If the emergency is over, and the alarm siren is no longer required, deactivation can take place through the device of the individual. Alternatively, the alarm siren calls out an automated message, instead of providing a default alarm sound, to help bystanders know to exit the area, thus facilitating rescue of the user. The alarm siren is much louder and more visible than the alert system that regularly comes with the smart band 21, and provides a sound to be heard by a bystander in an emergency. The alarm siren is configurable to have a specific sound and frequency, or automated message. The single LED light is a standard LED light with a gel cover of red, to project a color of red. The alarm siren and the single LED light are powered a plurality of batteries (not shown) separate from the battery of the smart band 21. The batteries also have a battery life that is visible on the screen of the smart band 21, unlike the battery life of the battery of the smart band 21, which is only visible on the smart band 21 itself. The batteries need to be replaced after prolonged use, and the user needs to determine when to replace the batteries. The alarm siren also needs to be changed after prolonged use, due to cellular pairings of the device of the individual, enabling the individual to be notified if a drowning is taking place.

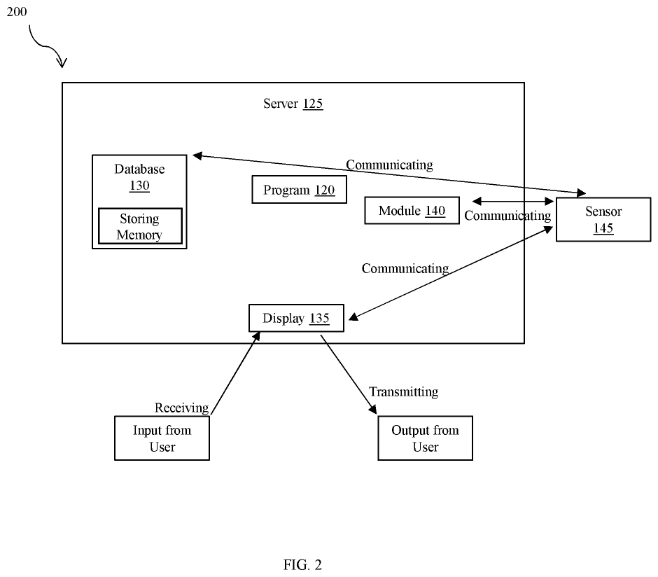

In an embodiment, illustrated in FIG. 2, a program 120 connects to the smart band 21 for monitoring heart rate, and operates on a server 125 over a network 200. The server 125 includes a database 130 for storing memory, a display 135 for both receiving input and transmitting output from a user, and a module 140 for allowing communication between the program 120 and a sensor 145 in the smart band 21. The sensor 145 takes measurements of temperature, oxygen levels, and depth of the user relative to water to aid determining baseline values, which are associated with safe swimming. The obtained values are sent to the database 130, the display 135, and the module 140 by the smart band 21. The program 120 functions on various devices, including but not limited to, a computer or a smart phone.

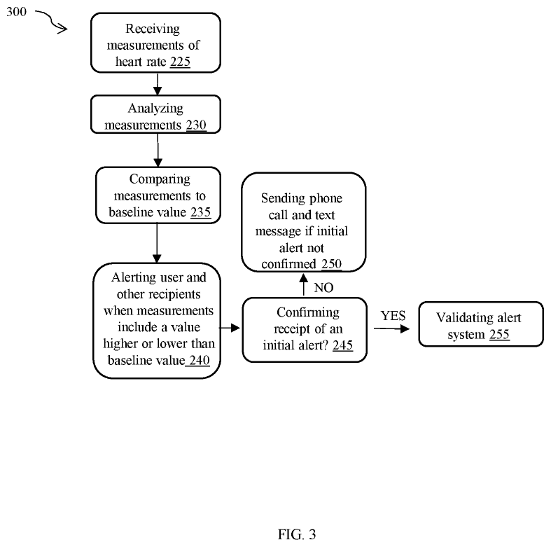

In one example, the smart band 21 performs flowchart 300 (FIG. 3) when monitoring a heart rate via a processor that executes instructions stored on a non-transitory computer readable medium. The smart band 21 receives measurements of the heart rate (and other measurements) from a user in step 225. The smart band 21 analyzes the measurements in step 230 and compares the measurements to a baseline value in step 235. The baseline value is calculated via SMART methods through the device of the individual. Examples of calculation methods include KARVONEN formula (220-(age)), which is a formula used by athletes to measure their approximate maximum BPM. The individual has to indicate an age of the user wearing the smart band 21, and an app will calculate a “zone” at which the user may be at risk. A spike in heart rate may be seen if, for instance, the user slips at an edge of a pool or has a sudden burst of physical activity. By contrast, an increase in heart rate is sustained if the user is at risk of a potential drowning. Slipping, for instance, may increase heart rate to beyond maximum BPM, but heart rate will return to normal very quickly. If the user is panicking, however, heart rate will increase and remain increased. Such differences are determined by the smart band 21, and entered into a profile of the user by the individual. The smart band 21 alerts the user and other recipients in step 240 (i.e. via an initial alert) when the measurement includes a value within a range higher or lower than the baseline value. Confirmation is processed as the initial alert by selecting a prompt (e.g. an “ok” prompt) that appears on a screen of an external device (i.e. a phone or a computer). The smart band 21 determines if there is confirmation of receipt of an initial alert in step 245. The smart band 21 determines if a recipient confirms or does not confirm receipt of the initial alert within a predetermined period of time (for example, 7 seconds) of the initial alert. If no confirmation takes place, then the smart band 21 instructs the server 125 to send a phone call and a text message to the recipient in step 250. If confirmation does take place, then the smart band 21 triggers machine learning (ML) techniques to validate the alert system of the smart band 21 in step 255.

The initial alert is confirmed if the recipient does one or more of the following actions: (a) places his or her finger over the initial alert; (b) a pressure level exceeds a first threshold with which the finger is placed over the initial alert; or (c) an amount of time that the recipient views the message exceeds a second threshold.

For example, if the pressure level is very low and thus below the first threshold, this may be indicative that the recipient has merely glossed over the initial alert and did not truly see the initial alert.

In another example, if the recipient views the message for a time below the second threshold, this may also be indicative that the recipient has merely glossed over the initial alert and did not truly see the initial alert.

Therefore, if measured actions by the recipient are below the first threshold and the second threshold, then the smart band 21 determines confirmation has not occurred even when the initial alert was technically seen by the recipient. The ML techniques record the pressure levels and the amount of time that the message is viewed. These are used as criteria to determine whether the alerts are effectively notifying the recipient.

The machine learning (ML) techniques validate the measured baseline values, deviations from the baseline values (e.g., higher or lower than the baseline value), and alerting measures as having: (i) deterministic capability in determining if a drowning or a heart attack is occurring; and (ii) notifying others that the user is drowning or having a heart attack. Stated another way, not all users exhibit the same baseline values and deviations from the baseline values during drowning. Thus, a personalized baseline value indicative of safe swimming or normal heart function is determined by the smart band 21. Additionally, not all recipients may respond the same way to notifying others if the smart band 21 determines that the user may be drowning or having a heart attack, based on deviations from the baseline value. Accordingly, the ML techniques account for the lack of uniformity with respect to measured values associated with normal heart rates and drowning, and whether recipients respond to messages and alerts. The ML techniques can in turn modify aspects of the message, such as making the message brighter or emitting a loud sound to indicate that the user is drowning or having a heart attack. This ensures that each user wearing the smart band 21 receives a customized assessment of the user's heart rate and other health parameters, while sufficient notification measures are sent.

The ML techniques herein use a neural network. The neural network can include any combination of neural networks including, but not limited to, Perception Neural Network, Feed Forward Neural Network, Artificial Neuron, Deep Feed Forward Neural Network, Radial Basis Function Neural Network, Recurrent Neural Network, Long/Short Term Memory, Gated Recurrent Unit, Auto Encoder Neural Network, Variation AE Neural Network, Demising AE Neural Network, Sparse AE Neural Network, Markov Chain Neural Network, Modular Neural Network, Hopfield Network, Boltzmann Machine, Restricted BM Neural Network, Deep Belief Network, Deep Convolution Network, Deconvolutional Network, Deep Convolution Inverse Graphics Network, Generative Adversarial Network, Liquid State Machine Neural Network, Extreme Learning Machine Neural Network, Echo State Network, Deep Residual Network, Kohonen Self Organizing Neural Network, Support Vector Machine Neural Network, Neural Turing Machine Neural Network, Convolution Neural Networks such as LeNet, AlexNet, ZF Net, GoogLeNet, VGG Net, Microsoft ResNet, and Region Based CNNs including, but not limited to, Fast R-CNN, Faster R-CNN, R-FCN, Multibox, SSD, and YOLO, amongst others which are known to those skilled in the art.

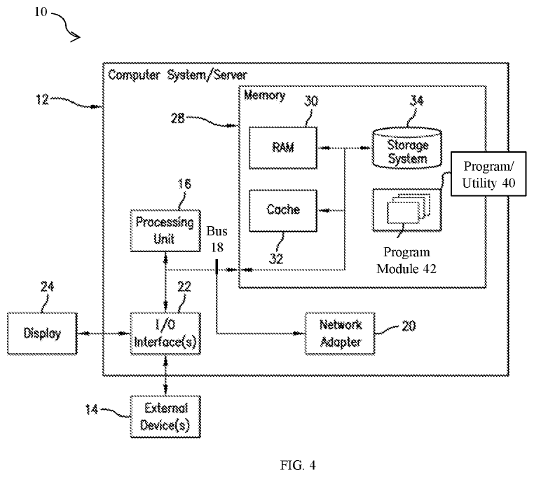

As shown in FIG. 4, a computer system/server 12 in a computing node 10 is shown in the form of a general-purpose computing device. Components of the computer system/server 12 include, but are not limited to, one or more processors or processing units 16, a system memory 28, and a bus 18 that couples various system components including system memory 28 to processor 16.

The bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The computer system/server 12 typically includes a variety of computer system readable media. Such media may be any available media that is accessible by computer system/server 12, and it includes both volatile and non-volatile media, removable and non-removable media.

The system memory 28 can include computer system readable media in the form of volatile memory, such as random access memory (RAM) 30 and/or cache memory 32. The computer system/server 12 may further include other removable/non-removable, volatile/non-volatile computer system storage media. By way of example only, the storage system 34 is provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM, or other optical media can be provided. In such instances, both types of disk drives are connected to the bus 18 by one or more data media interfaces. As will be further depicted and described below, the system memory 28 may include at least one program product having a set (e.g., at least one) of program modules that are configured to carry out the functions of embodiments of the invention.

In an embodiment, a program/utility 40, having at least one program module 42, is stored in the system memory 28. An operating system, one or more application programs, other program modules, and program data may also be included. Each of the operating system, one or more application programs, other program modules, and program data or some combination thereof, may include an implementation of a networking environment. The program module 42 generally carries out functions and/or methodologies of embodiments of the invention as described herein.

In another embodiment, the computer system/server 12 also communicates with one or more external devices 14 such as a keyboard, a pointing device, and a display 24. Other devices include one or more devices that enable a user to interact with the computer system/server 12 and/or any devices (e.g., network card, modem, etc.) that enable the computer system/server 12 to communicate with one or more other computing devices. Such communication can occur via at least one Input/Output (I/O) interface 22. Still yet, the computer system/server 12 can communicate with one or more networks such as a local area network (LAN), a general wide area network (WAN), and/or a public network (e.g., the Internet) via a network adapter 20. As depicted, the network adapter 20 communicates with the other components of the computer system/server 12 via the bus 18. It should be understood that although not shown, other hardware and/or software components could be used in conjunction with computer system/server 12. Examples, include, but are not limited to, microcode, device drivers, redundant processing units, external disk drive arrays, RAID systems, tape drives, and data archival storage systems.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be, for instance: assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++, and conventional procedural programming languages, such as the “C” programming language or similar programming languages. The computer readable program instructions may execute entirely on a computer of the user, partly on the computer of the user, as a stand-alone software package, partly on the computer of the user, and partly on a remote computer or entirely on the remote computer or a server. In the latter scenario, the remote computer may be connected to the computer of the user through any type of network, including a local area network (LAN) or a wide area network (WAN). The connection may also be made to an external computer (for example, through the Internet using an Internet Service Provider). In some embodiments, electronic circuitry includes, for example, programmable logic circuitry, field-programmable gate arrays (FPGA), or programmable logic arrays (PLA) that may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It is understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart, block diagrams, and/or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart, block diagrams, and/or blocks.

The computer readable program instructions may also be loaded onto a computer, other programmable data processing apparatus, or other device to cause a series of operational steps to be performed on the computer, other programmable apparatus or other device, to produce a computer implemented process (such that the instructions which execute on the computer, other programmable apparatus, or other device), to implement the functions/acts specified in the flowchart, block diagrams, and/or blocks.

The flowchart and block diagrams in the FIGS. illustrate the architecture, functionality, and operation of possible implementations of systems, methods, and computer program products according to various embodiments of the present invention. In this regard, each step in the flowchart or each block in the block diagrams may represent a module, segment, or portion of instructions, which comprises one or more executable instructions for implementing the specified logical function(s). In some alternative implementations, the functions noted in the block may occur out of the order noted in the FIGS. For example, two blocks shown in succession may, in fact, be executed concurrently, or the blocks may sometimes be executed in reverse order, depending upon the functionality involved. It is also noted that each block of the block diagrams and/or steps of the flowchart illustration, and combinations of blocks in the block diagrams and/or steps in the flowchart illustration, can be implemented by special purpose hardware-based systems that perform the specified functions/acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical applications, or technical improvement over technologies found in the marketplace, or to enable others of ordinary skill in the art to understand the embodiments disclosed herein.

What is claimed is:

1. A smart band, for alerting of a user in an emergency, comprising:
a bracelet to be worn by the user;
a screen disposed along the bracelet to display alerts and messages;
an alert system for notifying that the user is in an emergency;
wherein the screen is powered by a battery having a battery life;
wherein the band includes technology compatible with an application on a phone or a computer;
a sensor for measuring heart rate;
wherein the smart band is configured to monitor a heart rate via a processor that executes instructions stored on a non-transitory computer readable medium, based on measurements received by the heart rate sensor, and analyze the measurements and determine whether a user is in a state of emergency and then send an alert if the user is in a state of emergency.

2. The smart band of claim 1, further comprising a plurality of LED lights, disposed on the screen, that when lit, correspond to a percentage of the battery life of the battery;
wherein the screen displays a warning message when the percentage of the battery life drops to a particular amount;
wherein the screen is configured to indicate how much time the battery is operable the battery being connectable to a charger for replenishing the battery life of the battery.

3. The smart band of claim 1, further comprising a first list identifying personal contacts and a second list identifying emergency contacts, both stored on the screen;
wherein the first list and the second list connect to a personal application and a corporate application, respectively, on a separate device.

4. The smart band of claim 3, further comprising an alarm siren and a single LED light synced to both the personal application and the corporate application;
wherein the alarm siren is louder and more visible than the alert system, and provides a sound to be heard by a bystander during the emergency.

5. The smart band of claim 4, wherein both the alarm siren and the single LED light are powered a plurality of batteries separate from the battery.

6. The smart band of claim 1, wherein the bracelet has a back surface having a barcode to identify the bracelet, and a locking mechanism that prevents the user from removing the bracelet;
wherein the locking mechanism is configured to be unlocked by a tool to allow the bracelet to be removed.

7. The smart band of claim 1, wherein the smart band is configured to analyze the heart rate measurements and compare the measurements to a predetermined baseline value and a heart rate zone within which the user may be at risk; wherein the smart band is configured to send an alert to an external computing device for alerting another individual, if the heart rate measurement is sustained within the zone for at least a predetermined period of time.

8. The smart band of claim 7, wherein the smart band is configured to determine if there is confirmation of receipt of an alert sent to the external computing device, and the smart band also determines if a recipient confirms or does not confirm receipt of the alert within a predetermined period of time of the alert; wherein if no confirmation takes place, then the smart band instructs a server to send a phone call and a text message to the another individual; wherein if confirmation does take place, then the smart band validates the alert system of the smart band.

9. The smart band of claim 8, further comprising a GPS tracker for identifying and displaying a location of the user to the computing device of the another individual; wherein the smart band is configured to send the location of the user based on data provided by the GPS tacker.

10. A smart band, for alerting of a user in an emergency, comprising:
a bracelet to be worn by the user;
a screen disposed along the bracelet to display alerts and messages;
wherein the screen is powered by a battery having a battery life;
wherein the screen is configured to display a warning message when the percentage of the battery life drops to a particular amount;
wherein the screen is configured to indicate how much time the battery is operable the battery being connectable to a charger for replenishing the battery life of the battery.
a first list identifying personal contacts and a second list identifying emergency contacts, both stored on the screen;
wherein the first list and the second list connect to a personal application and a corporate application, respectively, on a separate device.
a sensor for measuring heart rate;
a plurality of LED lights, disposed on the screen, that when lit, correspond to a percentage of the battery life of the battery;
an alert system for notifying that the user is in an emergency;
wherein the band includes technology compatible with an application on an external computing device;
an alarm siren and a single LED light synced to both the personal application and the corporate application;
wherein the alarm siren is louder and more visible than the alert system, and provides a sound to be heard by a bystander during the emergency;
a GPS tracker for identifying and displaying a location of the user to the computing device of the another individual; wherein the smart band is configured to send the location of the user based on data provided by the GPS tacker.
wherein the smart band is configured to monitor a heart rate via a processor that executes instructions stored on a non-transitory computer readable medium, based on measurements received by the heart rate sensor, and analyze the measurements and determine whether a user is in a state of emergency and then send an alert if the user is in a state of emergency;
wherein the smart band is configured to analyze the heart rate measurements and compare the measurements to a predetermined baseline value and a heart rate zone within which the user may be at risk; wherein the smart band is configured to send an alert to an external computing device for alerting another individual, if the heart rate measurement is sustained within the zone for at least a predetermined period of time;
wherein the smart band determines if there is confirmation of receipt of an alert sent to the external computing device, and the smart band also determines if a recipient confirms or does not confirm receipt of the alert within a predetermined period of time of the alert; wherein if no confirmation takes place, then the smart band instructs a server to send a phone call and a text message to the another individual; wherein if confirmation does take place, then the smart band validates the alert system of the smart band;
wherein both the alarm siren and the single LED light are powered a plurality of batteries separate from the battery;
wherein the bracelet has a back surface having a barcode to identify the bracelet, and a locking mechanism that prevents the user from removing the bracelet; wherein the locking mechanism is configured to be unlocked by a tool to allow the bracelet to be removed.

11. A method for determining if a user is in an unsafe situation, comprising the steps of:
receiving measurements of a heart rate from the user;
analyzing the measurements;
comparing the measurements to a baseline value; and
alerting the user and other recipients when the measurements include a value within a range higher or lower than the baseline value.

12. The method of claim 11, further comprising confirming receipt of an initial alert by the other recipients by selecting a prompt that appears on a screen of an external device.

13. The method of claim 11, further comprising the steps of sending a phone call and a text message to the other recipients if receipt of the initial alert is not confirmed within a predetermined period of time of the initial alert.

14. The method of claim 11, wherein alerting the user and other recipients when the measurements include a value within a range higher or lower than the baseline value, further comprises: utilizing machine learning techniques to validate the measurements as being an accurate basis for determining the unsafe conditions.

15. The method of claim 11, wherein the unsafe conditions comprises: drowning, abnormal heart rates, abnormal breathing rates, or a combination thereof.
